(19) United States
(12) Patent Application Publication
(10) Pub. No.: US 2006/0210645 A1
(43) Pub. Date: Sep. 21, 2006

(54) Pharmaceutically acceptable carrier for ophthalmic compositions

(76) Inventors: Charles P. Du Mee, Foster City, CA (US); Gene Barnett, San Francisco, CA (US); Michael Coy, Honolulu, HI (US)

Correspondence Address: REED SMITH LLP, 1301 K STREET, N.W., SUITE 1100 EAST TOWER, WASHINGTON, DC, 20005, US

(21) Appl. No.: 11/366,000

(22) Filed: Mar. 2, 2006

Related U.S. Application Data
(60) Provisional application No. 60/657,409, filed on Mar. 2, 2005.

(51) Int. Cl. A61K 33/42; A61K 31/717; A61K 31/66; A61K 31/56; A61K 31/57

(52) U.S. Cl. 424/601; 514/57; 514/130; 514/170

(57) ABSTRACT

A pharmaceutically acceptable carrier for ophthalmic compositions useful in treating or alleviating the symptoms of dry eye syndrome, elevated intra-ocular pressure, age-related maculopathy or age-related macular degeneration.

### FIELD OF THE INVENTION

The present invention relates generally to therapeutic compositions and methods of treatment in an ophthalmologic field, and more particularly, to ophthalmic pharmaceutical compositions useful by themselves as artificial tear compositions, or as carriers for active ingredients, particularly those useful for treating and/or preventing the ophthalmologic clinical symptoms and/or signs associated with dry eye syndrome, elevated intraocular pressure, age-related maculopathy and age-related macular degeneration.

### BACKGROUND OF THE INVENTION

Broadly speaking, dry eye syndrome is a disorder of the tear film due to tear deficiency or excessive tear evaporation which causes damage to the interpalpebral ocular surface and is associated with symptoms of ocular discomfort. (Lemp, M. A. Report of the National Eye Institute/Industry Workshop on Clinical Trials in Dry Eyes, The Contact Lens Association of Ophthalmologists Journal, 21(4):221 -231 (1995)). Findings show differences between Sjögren's associated keratoconjunctivitis sicca (KCS) and non-Sjögren's KCS. (Nelson, J. D., et al., Cellular Acetate Impressions of the Ocular Surface: Dry Eye States, Arch Ophthalmol., 101:1869-1982 (1983); Tseng, S. C. G. Staining of Conjunctival Aquamous Metaplasia by Impression Cytology, Ophthalmol., 92:728-733 (1985); Pflugfelder, S. C., et al., Cytological Features of Primary Sjögren's Syndrome, Ophthamol., 97:985-991 (1990)). Neurotransmitters (Mircheff, A. K., et al., Autoimmunity of the Lacrimal Gland in the Dry Eye,. Internat. Ophth, Clinics, 34(1):1-18 (1994); Mircheff, A. K., et al., Understanding the Causes of Lacrimal Insufficiency: Implications for Treatment and Prevention of Dry Eye Syndrome, Res. Prev. Blindness Sci. Writers' Seminar, 51-54 (1993)), viruses (Mircheff, A. K., et al., Understanding the Causes of Lacrimal Insufficiency: Implications for Treatment and Prevention of Dry Eye Syndrome, Res. Prev. Blindness Sci. Writers' Seminar, 51-54 (1993)), and hormones (Mircheff, A. K., et al., Understanding the Causes of Lacrimal Insufficiency: Implications for Treatment and Prevention of Dry Eye Syndrome, Res. Prev. Blindness Sci. Writers' Seminar, 51-54 (1993); Sullivan, D. A: Ocular Mucosal Immunity, in Handbook of Mucosal Immunology. Academic Press, 47:569-597 (1994)) are important in regulating tear production and immune activity in the lacrimal glands and the ocular surface. Also, meibomian gland dysfunction can increase tear evaporation with an increase in tear film osmolarity and resultant ocular surface disease. (Mathers, W. P., et al., Meibomian Gland Dysfunction in Chronic Blepharitis. Cornea, 111:763-765 (1991)).

Tear film quality depends on fine regulatory mechanisms affected by neuronal and hormonal influences. Indeed, receptors for androgens, estrogens, progesterone and prolactin have been identified in several ocular tissues in the rat, rabbit and in humans. These hormones regulate the immune system, the morphology and secretory functions of lacrimal glands and the functioning of Meibomian glands. The influence of hormone replacement therapy in menopausal women remains unclear, as some authors support the idea that hormones improve the quality and the volume of tear film, whereas others have argued that they increase the risk of dry eye. Finally, knowledge of the interactions between the hormones that influence the function of the lacrimal gland is an essential element for the understanding of the regulation of lacrimal gland function. Additional data suggest that optimal bioavailable androgen levels are essential for normal lacrimal gland function and that prolactin and estrogens also play important roles in providing a hormonal milieu that contributes to normal lacrimal gland function. ( Oprea, L., Tiberghien, A., Cruezot-Garcher, C., Baudouin, C., Hormonal Regulatory Influence in Tear Film, J. Fr. Ophtalmol., Oct. 27(8):93341 (2004)).

The standard treatment of KCS with artificial lubricants provides temporary symptomatic relief. While there has been described treatment of post menopausal females with dry eye syndrome using oral Premarin therapy, the oral or parenteral administration of estrogen can frequently produce side effects such as vaginal bleeding, breast tenderness and other undesired effects, and the therapeutic effects derived from oral therapy are minimal. This result is now understood, a least in part, to flow from the fact that there are very few estrogen receptors in the conjunctiva relative to other tissues of the body. (Gans, L. A., et al., Estrogen And Progesterone Receptors and Human Conjunctiva, Am. J. Ophthalmol., 109(4):474477 (1990). Further, such oral or parenteral administration implicates the entire body structure in an indeterminate effort to secure an effect in a localized area (the eye). Conservative medicine would indicate the desirability of a local or topical rather then systemic drug administration, thus limiting the effect of the hormone to the target site.

The ideal method of accomplishing this is through the use of topically applied pharmaceutical agents in drop form. See for example U.S. Pat. No. 6,096,733.

Conventional ophthalmic solutions used generally in the ophthalmologic field are easily portable, and therefore ophthalmic solutions, such as those containing sodium hyaluronate, have been commercialized for treating the ophthalmologic clinical symptoms and signs in Sjogren syndrome.

Further studies since about 1990 have shown that estrogen is a component of human tears and that it may play a role in ophthalmic changes in ocular tissue. (Kramer, P. et al., Cyclic Changes in Conjunctival Smears from Menstruating Females, Ophthalmol., 97:303-307 (1990); Metka, M. et al., Ophthalmic complaints as a climacteric symptom, Maturitas, 14:3-8 (1991)). Other studies, even more recently, have intimated that post-menopausal patients given low systemic doses of estriol (a hydroxylated form of 17-β-estradiol) at a dose of 0.25 mg per day, or that even near homeopathic concentrations of 17-β-estradiol (0.00025%) in drops applied every 6 hours (in women already taking 2 mg estriol valerate daily by mouth) gave varying or marginal improvement in corneal lens transmittance and autofluorescence. (de Castillo, Benitez, et al., Effects of Estrogen Use on Lens Transmittance in Postmenopausal Women, Ophthalmol., 104:970-973 (1997)).

U.S. Pat. No. 6,107,289; teaches an approach for management of KCS, especially as manifested in Sjögren's syndrome, involving the topical application to the eye of a preparation containing a therapeutic amount of an androgen or androgen analogue, at a dose rate of less than 1 mg/day.

The present invention is concerned with formulating an optimal pharmaceutical carrier for ophthalmic delivery of active ingredients, particularly estrogens and androgens for the treatment of symptoms of dry eye, elevated intraocular pressure, age-related maculopathy and age-related macular degeneration. In the absence of active ingredients, the pharmaceutical carrier of the present invention can also be used prophylactically to alleviate or ameliorate ocular symptoms, particularly those present in dry eye syndrome. Further, in the absence of active ingredients, the pharmaceutical carrier of the present invention can also be used as an over-the-counter artificial tear, and is also formulated to ease discomfort and irritation associated with contact lenses.

### SUMMARY OF THE INVENTION

Accordingly, the present invention provides a composition useful as a pharmaceutical carrier for ophthalmic applications, that is particularly suitable as a topical delivery vehicle for water soluble pharmaceutically active ingredients, particularly estrogens and androgens, separately or in combination, useful in the alleviation of symptoms of dry eye syndrome, age-related maculopathy, age-related macular degeneration or elevated intraocular pressure.

The compositions of the present invention are particularly suitable for use in an ophthalmic pharmaceutical composition providing a satisfactory therapeutic and/or prophylactic effect on the ophthalmologic clinical symptoms of dry eye syndrome, while being useful and safe for extended use. The compositions are particularly well suited for formulation into an ophthalmic solution, ointment, and eyewash.

In addition to their utility as a pharmaceutical carrier or vehicle for other active ingredients, the compositions of the invention can be used by themselves as over-the-counter artificial tear compositions, or wetting agents. The compositions of the present invention provide an artificial tear composition that is contact lens tolerant, and can be used to ease the irritation and discomfort associated with contact lenses. Further, the compositions of the invention alone (i.e., without added active ingredient) have been found to alleviate some of the clinical symptoms associated with dry eye syndrome.

The beneficial attributes of the compositions of the invention were discovered while studying hormonal substances which have satisfactory therapeutic and/or prophylactic effect on the ophthalmologic clinical symptoms and signs in dry eye syndrome and elevated intraocular pressure. During this investigation, the present inventors unexpectedly found that placebo patients utilizing the pharmaceutical carrier formulation of the present invention, without the active ingredients, widely reported alleviation of their clinical symptoms in dry eye syndrome without any side effect even after administration for a relatively-long time.

In general, the compositions of the present invention provide a pharmaceutical carrier or delivery vehicle: a) having a sterile, buffered isotonic solution, b) containing mucin-like substances that tend to increase the contact time between the active drug substances and the eye surface, and c) that are preferably free of benzalkonium chloride, which is a cationic surfactant that is known to be incompatible in solutions with steroid sodium phosphate salts. More particularly, the preferred delivery vehicle comprises, in single use or multi-use vials as applicable, an aqueous solution having a pH within the range of 4-8, preferably pH 6-8. This aqueous solution preferably contains dibasic sodium phosphate, sodium chloride, edetate disodium, povidone, poloxamer 188, polyethylene glycol, hydroxy ethyl cellulose, purified water, hydrochloric acid or sodium hydroxide for pH adjustment, and optionally, methylparaben and/or propylparaben and/or phenoxyethanol as preservatives. It is further contemplated that this aqueous formulation can also be used in formulating a liposomal delivery vehicle as well. Such compositions may be particularly compatible with the therapeutic needs of contact lens users.

In addition to use of the compositions of the present invention as artificial tears or wetting agents, it is further contemplated that such compositions can be used as carriers for ophthalmic active ingredients. For example, the compositions of the invention provide a suitable pharmaceutical carrier composition for delivery of estradiol and/or androgens useful in the treatment and or prevention of symptoms of dry eye syndrome and elevated intraocular pressure. In particular, the compositions of the present invention can be used to formulate a pharmaceutical composition comprising 17-β-estradiol or its esters (for example the 3-phosphate disodium salt) and its water-soluble, storage-stable derivatives (β-estradiol glucuronide β-estradiol hemisuccinate, β-estradiol phosphate, β-estradiol sulfate and their 3, 17 diesters, 17 monoesters and 3 monoesters) and/or androgens or androgen analogues, (hereinafter collectively referred to as “androgens”) such as 17-α-methyl-17-β-hydroxy-2-oxa-5α-androstan-3-one, 4,5α-dihydrotestosterone derivatives, testosterone derivatives, 19-nortestosterone derivatives, 17β-hydroxy-5α-androstane derivatives containing ring A unsaturation, their esters, and their cationic or phosphorylated derivatives, designed to increase solubility in hydrophilic media.

It is contemplated that the pharmaceutical carrier of the present invention, with or without the active ingredients, can be used to ameliorate the symptoms of dry eye syndrome in post-menopausal women, women who have had oophorectomies or total hysterectomies or premature ovarian failure, and pre-menopausal women with hormonal abnormalities including insufficient estrogen production.

It is to be understood that both the foregoing general description and the following detailed description are exemplary and explanatory and are not intended to limit the invention as claimed. Other objects and features of the invention will become apparent from the following detailed description. All references cited in the instant disclosure are incorporated herein by reference.

### DETAILED DESCRIPTION OF THE PREFERRED EMBODIMENTS OF THE PRESENT INVENTION

The compositions of the invention provide formulations suitable for use as artificial tears and ocular welling agents, as well as vehicles for the delivery of therapeutically active ingredients. In a preferred embodiment, the composition is an aqueous solution comprising, on a weight percent basis, about:

| | |
|---|---|
| Dibasic sodium phosphate, USP | 0.05-1.0% |
| Sodium Chloride, USP | 0.2-0.9% |
| Edetate disodium, USP | 0.05-1.0% |
| Povidone, USP | 0.05-2.0% |
| Poloxamer NF | 0.001-0.05% |
| Polyethylene glycol | 0.05-1.0% |
| Hydroxyethyl Cellulose NF | 0.05-1.0% |
| Purified water, USP, | q.s to 100% |
| HCl or NaOH | to adjust pH to pH 6-8 |

A more preferred composition of the invention comprises:

| | |
|---|---|
| Dibasic sodium phosphate, USP | 0.3% |
| Sodium Chloride, USP | 0.6% |
| Edetate disodium, USP | 0.1% |
| Povidone, USP | 0.37% |
| Poloxamer NF | 0.004% |
| PEG | 0.12% |
| Hydroxyethyl Cellulose NF | 0.2% |
| Purified water, USP, | q.s to 100% |
| HCl or NaOH | to adjust pH to pH 6-8. |

The preferred Povidone is K-15, or K-17, with K-17 being particularly preferred. The preferred Poloxamer is Poloxamer 188. The preferred polyethylene glycol is PEG 3350, and the preferred hydroxyethyl cellulose is Hydroxyethyl Cellulose 100.

The composition may optionally comprise one or more preservatives such as methylparaben, NF, and/or propylparaben, NF, and/or phenoxyethanol each of which may be present in an amount ranging from about 0.005 to about 0.5% by weight. The preferred composition comprises both 0.04 weight percent methlyparaben and 0.02 weight percent propylparaben.

This pharmaceutical carrier may be used by itself as a topical composition for alleviation of symptoms associated with dry eyes and elevated intraocular pressure. However, it is preferably used as a carrier for delivery of ophthalmically active ingredients, and is most preferably used as a carrier for delivery of estrogen and/or androgen analogues useful in the treatment of dry eye syndrome and elevated intraocular pressure.

Particularly preferred active ingredients compoundable with the pharmaceutical carrier of the present invention are derivatives of estrogen known as 17-β-estradiol (or the 3-phosphate disodium salt) and its water-soluble, storage-stable derivatives (β-estradiol glucuronide β-estradiol hemisuccinate, β-estradiol phosphate, β-estradiol sulfate and their 3,17 diesters, 17 monoesters and 3 monoesters) and/or one or more androgens, preferably selected from the group consisting of 17-α-methyl-17-β-hydroxy-2-oxa-5α-androstan-3-one, 4,5α-dihydrotestosterone derivatives, testosterone derivatives, 19-nortestosterone derivatives, 17β-hydroxy-5α-androstane derivatives containing ring A unsaturation, their esters, and their cationic or phosphorylated derivatives, designed to increase solubility in hydrophilic media. Particularly preferred androgens are 17-α-methyl-17-β-hydroxy-2-oxa-5α-androstan-3-one, 4,5α-dihydrotestosterone and phosphorylated derivatives. Particularly preferred pharmaceutically active substances for use with the pharmaceutical carrier of the present invention are those that are derivatized to have enhanced solubility and stability at essentially neutral pH 6-8 (though the pH is not absolutely critical and could suitably range between 4-8)

The amount of active ingredient that is to be admixed with the carrier depends on the use and factors such as the age of the patient, the particular condition to be treated, the frequency of administration, and the means of administration. The concentration of active ingredients can range from about 0.001 percent to about 10 percent by weight. In a most preferred embodiment, the concentration of 17-β-estradiol 3-phosphate disodium is in the range of 0.01-1.0 weight percent. In another preferred embodiment, an androgen is present in a concentration of about 0.001 to about 0.1 percent by weight of the composition.

In an alternate embodiment, it is contemplated that a sterile ophthalmic solution of ophthalmically active agent can be comprised of a liposomal drug delivery system whose aqueous phase comprises the pharmaceutical carrier of the present invention. Liposomal therapy has been successfully used in ophthalmology not only for pre- and postoperative antisepsis, but also for the treatment of bacterial and viral conjunctivitis and for prophylaxis against ophthalmia neonatorum. (Margalit R., Liposome-Mediated Drug Targetin in Topical and Regional Therapies, Crit. Rev. Ther. Drug Carrier Syst., 12(2-3):233-61 (1995)). A Method for formulating such a product can be found in U.S. Pat. No. 5,662,931, which is herein incorporated by reference.

### EXAMPLE 1

A. Preparation of Compositions of the Invention.

The following is a description of a filling, labeling and packaging procedure for the preparation of ophthalmic dropper bottles in accordance with a preferred embodiment of this invention. First the bottles are inspected and must meet the following requirements; matches the physical description in the vial specification, presence of a certificate of analysis and presence of preparation records. The vials are then released for manufacturing use.

The dropper tips are inspected and must meet the following requirements; matches the physical description in the dropper tip specification, presence of a certificate of analysis and presence of preparation records. The dropper tips are then released for manufacturing use.

The caps are inspected and must meet the following requirement: matches the physical description in the cap specification. The caps are then released for manufacturing use.

A line clearance of the work area is performed prior to and at the end of each batch of the filling operation. All primary components are sterilized prior to use.

Several of the raw materials are prepared as three separate pre-mixed solutions prior to combining to produce the final solution. Pre-Mix hydroxy ethyl cellulose (“HEC”): The day before the filling operation, the HEC 100 is slowly dispersed in water and mixed until the polymer is completely hydrated and dissolved. Pre-Mix Salts: Sodium phosphate dibasic, sodium chloride and Edetate (“EDTA”) disodium are added to water and mixed until dissolved. Optionally Pre-Mix Parabens: Purified water is heated to 50±5° C. The methylparaben and propylparaben are added and mixed until completely dissolved. The solution is added to the final mixing tank. Purified water is added to the mixing tank. The Pre-Mix Salt solution is added and mixed and then the Pre-Mix Paraben solution is added and mixed. The solution is cooled to below 30° C. Povidone K-17, Poloxamer 188 and PEG 3350 are then added one at a time and mixed, ensuring that each ingredient is completely dissolved and the solution is clear before the next one is added. The Pre-Mix HEC solution is then added, and mixed well until the solution is clear and the pH is measured.

If an active ingredient is to be used, the batch of active ingredient is first assayed for content, added slowly and mixed until completely dissolved and the pH is measured. The pH is adjusted to about 6.0 to 8.0, if necessary, with NaOH or HCl.

The compounded solution is filtered through redundant 0.22 μm filters into a pre-sterilized surge vessel. The solution is filled into pre-sterilized ophthalmic dropper bottles. Each bottle is capped and sealed with a pre-sterilized dropper tip and cap before removal from the clean area.

Unlabeled bottles are 100% inspected. The bottles are labeled with the date, product name, product code and the lot number. The bottles are then stored in quarantine until final QC testing and QA release.

B. Selection/Preparation of Active Ingredients.

The method of synthesis of 17-β-estradiol 3-phosphate disodium is reported in Acta Chem. Scan. 12, 1675-1689 (1958), which is incorporated herein by reference, and is briefly described as follows:

17-β-estradiol 17-acetate (Molecular Weight=314.4, Melting Point 220-224 ° C. and optical rotation 47° ) is phosphorylated in the presence of concentrated ortho-phosphoric acid (H₃PO₄) with heat and refluxing to yield the intermediate 17-β-estradiol 3-phosphate 17-acetate. The latter compound is selectively hydrolyzed in the presence of sodium bicarbonate in aqueous alcohol to yield sodium acetate and 17-β-estradiol 3-phosphate disodium. The desired steroid phosphate ester is recrystallized from dilute alcohol.

More information on the preparation and characteristics of estradiol phosphate is set forth in the article by Diczfalusy (Diczfalusy, E. High Molecular Weight Enzyme Inhibitors, Chemica Scandinavia, Vol.12 No. 8, pp. 1675-1689 (1958)) which is incorporated herein by reference.

Regarding the androgen, selection of the most appropriate therapeutic androgen will depend upon a given hormone's immunological activity, potential side effects and form of administration. For example, topical testosterone may be quite effective in reducing lacrimal inflammation, and its methylated analogue appears to have no toxic side effects on parameters such as intraocular pressure. (Knepper, P. A., Collins, J. A., and Frederick, R., Effect of Dexamethasone, Progesterone, and Testosterone on IOP and GAGs in the Rabbit Eye, Invest. Ophthalmol. Vis. Sci., 26:1093-1100 (1985)). However, a variety of other modified and/or anabolic androgens (Wilson, J. D., and Foster, D. W., eds., “Williams Textbook of Endocrinology,” WB Saunders Company, Philadelphia (1985), Vida, J. A., “Androgens and Anabolic Agents,” Academic Press, New York (1969)) may be more effective than testosterone.

In order to increase the aqueous solubility of the androgen, phoshorylated ester derivatives of the androgens are preferred and can be prepared by means commonly available in the art. For example, the most convenient method of synthesis of steroid esters is reaction of the steroid in a 2:1 mixture of pyridine and the anhydride of the desired ester: for example, propionic anhydride would be used to make the propionate ester. A large excess (at least 10 times) of the anhydride compared to the steroid would be required. This would then be purified by diluting with at least 10 parts of water to each part of pyridine, adding 1 part ether, decanting the water after shaking, and then washing with 10 parts water repeatedly in a separatory funnel. This would be followed preferably by recrystallization or chromatography for purification.

Androgens to be used include testosterone, dihydrotestosterone (also termed allodihydrotestosterone, androstanolone, stanolone, 5 alpha-dihydrostestosterone), fluoxymesterone, stanozolol, nortestosterone propionate, dehydroepiandrosterone (an androgen precursor, also termed androstenolone, dehydroisoandro-sterone, DHEA, transdehydroandrosterone), oxandrolone; methyldihydrotestosterone (also termed methylandrostanolone), oxymetholone, 5 alpha-androstan-17β-ol-3-oxime, 5 alpha-androstan-17 alpha-ol-3-one-acetate, (1) 2,(5 alpha)-androsten-17β-ol, 5 alpha-androstan-2 alpha-methyl-17β-ol-3-one, methyltestosterone, and their soluble ester derivatives.

These androgens are representative of the major structural subclasses of androgens, as disclosed in Vida (Vida, J. A., “Androgens and Anabolic Agents,” Academic Press, New York (1969), hereby incorporated by reference. The subclasses include (a) androgenic compounds with unusual structural features (e.g., 17 alpha-methyl-17β-hydroxy-2-oxa-5 alpha-androstan-3-one, also termed oxandrolone); (b) testosterone derivatives (e.g., methyltestos-terone); (c) 4,5 alpha-dihydrotestosterone derivatives (oxymetholone); (d) 17β-hydroxy-5 alpha-androstane derivatives containing a ring A unsaturation, excluding testosterone derivatives (e.g., 2,(5 alpha)-androsten-17β-ol); and (e) 19-nortestosterone derivatives (e.g., 19-nortestosterone propionate). It may be that certain structural features impart more optimal immunosuppressive characteristics, which would be of benefit in selecting specific androgens for human use.

Also, relative to standards (typically testosterone), these androgens include compounds displaying: (a) augmented androgenic (i.e., virilizing) activity coupled with an even larger increase in anabolic activity (e.g., fluoxymesterone); (b) enhanced anabolic action with unchanged androgenic effects (e.g., oxymetholone, dihydrotestosterone); (c) decreased androgenic ability with unchanged anabolic activity (e.g., 19-nortestosterone propionate); and (d) decreased androgenic capacity paralleled by increased anabolic activity (e.g., oxandrolone, stanozolol). Preferred androgens for use in compositions of the invention are those which have far more anabolic, than virilizing effect, (e.g., oxandrolone possesses 322% of the anabolic and 24% of the androgenic activity of methyltestosterone (Vida, J. A., “Androgens and Anabolic Agents,” Academic Press, New York (1969)).

The pharmaceutical carrier by itself is useful in alleviating symptoms of dry eye syndrome. As such it may be used by itself as a placebo, a tear substitute, or otherwise with or without the presence of active ingredients. The carrier, without the active ingredients, is also useful in alleviating discomfort and minor irritation associated with the wearing of contact lenses.

Prior to an application of the pharmaceutical carrier of the present invention or a pharmaceutical composition comprising the pharmaceutical carrier and an active ingredient useful in the treatment of dry eye syndrome, it is necessary to establish the presence of dry eye syndrome in the test population and to follow its course under treatment. It is imperative that the diagnosis of dry eye syndrome be correct. Occasionally KCS is diagnosed by use of the Schirmer test. The Schirmer test, however, is not always the most accurate test. It consists of taking a strip of filter paper 30 mm long and 5 mm in length and placing it in the patient's lower conjunctival sac. After 5 minutes, the length of paper moistened is measured and used as an indicator of lacrimal fluid quantity. Factors such as temperature, humidity, lacrimal viscosity, types of filter paper used, batch variations between lots of paper, and other factors can significantly affect the data produced by this test.

The diagnosis of dry eye syndrome in the present invention, can be made on the basis of one or more of the following tests. Microscopic evaluation of the tear film with particular attention to the marginal tear strip, viscosity and debris content of the precorneal tear film, and lid examination may be performed. Staining the ocular surface with Rose Bengal or Lissamine Green, dyes which indicate cellular damage, Schirmer testing, tear osmolarity, measurement of tear break-up time (TBUT), may also be used. In addition, the maturation index (a Papanicolaou stained sample of conjunctival epithelium) may also be performed.

In the case of post-menopausal women, menopause is confirmed with follicle stimulating hormone and luteinizing hormone serum determinants and estradiol levels. Postmenopausal women with dry eye syndrome have have been shown to have lower estradiol levels (mean E₂ estradiol levels of 3.47 picograms/milliliter), than that of normal postmenopausal women (mean E₂ estradiol levels of 16.05 picograms/milliliter) (U.S. Pat. No. Re. 34,578, col. 2, Ln. 56-59).

The treatment regime will likely depend on numerous factors, and vary from person to person. For example, one or two drops per eye given two to four times a day may be used, but application may also be more or less frequent. However, other alternative pharmaceutical modes of administration may be used—such as a slow release mode, or any other topical method, and the concentration may vary with individual response, as well as the treatment intervals and duration. Blood levels of the active hormone ingredients should also be determined and monitored.

All the references cited herein are hereby incorporated by reference. It will be apparent to those skilled in the art that various modifications and variations can be made to the present invention without departing from the spirit or scope of the invention. Thus, it is intended that the present invention cover the modifications and variations of this invention provided they come within the scope of the appended claims and their equivalents. Accordingly, the invention is not limited by the embodiments described above which are presented as examples only, but can be modified in various ways within the scope of protection defined by the appended patent claims.

We claim:

1. A therapeutic composition for topical ophthalmic application comprising, on a weight percent basis:

| | |
|---|---|
| Dibasic sodium phosphate | 0.05-1.0% |
| Sodium Chloride, | 0.2-0.9% |
| Edetate disodium | 0.05-1.0% |
| Povidone | 0.05-2.0% |
| Poloxamer | 0.001-0.05% |
| Polyethylene glycol | 0.05-1.0% |
| Hydroxyethyl Cellulose | 0.05-1.0% |
| Purified water, | q.s to 100% |
| HCl or NaOH | to adjust pH to pH 6-8. |

2. The composition of claim 1 comprising, on a weight percent basis, about:

| | |
|---|---|
| Dibasic sodium phosphate | 0.3%; |
| Sodium Chloride | 0.6%; |
| Edetate disodium | 0.1%; |
| Povidone K-17 | 0.37%; |
| Poloxamer | 0.004%, |
| Polyethlyene glycol | 0.12%; |
| Hydroxyethyl Cellulose | 0.2%, |
| Purified water | q.s to 100%; |
| HCl or NaOH | to adjust pH to pH 6-8. |

3. The composition of claim 1, further comprising one or more preservatives selected from the group consisting of methylparaben, propylparaben, benzalkonium chloride and phenoxyethanol.

4. The composition of claim 1, further comprising one or more hormones, or a derivative thereof, dissolved or suspended therein.

5. The composition of claim 4, wherein the hormone is 17-β-estradiol or a derivative thereof.

6. The composition of claim 5, wherein the hormone is 1 7-β-estradiol-3-phosphate.

7. The composition of claim 5, wherein said hormone is present in an amount of from about 0.001 to about 10.0% by weight.

8. The composition of claim 5, wherein said hormone is present in an amount of from about 0.001 to about 0.1% by weight.

9. The composition of claim 4, wherein the hormone is an androgen.

10. The composition of claim 4, wherein the one or more hormones comprises a combination of 17-β-estradiol, or a derivative thereof, and an androgen.

11. The composition of claim 9, wherein said androgen is selected from the group consisting of 17-β-hydroxy-2-oxa-5α-androstan-3-one, 4,5α-dihydrotestosterone, and their nitrogenated or phosphorylated derivatives 17-α-methyl-17-β-hydroxy-2-oxa-5α-androstan-3-one, 4,5α-dihydrotestosterone derivatives, testosterone derivatives, 19-nortestosterone derivatives, 17β-hydroxy-5α-androstane derivatives containing ring A unsaturation, their esters, and their cationic or phosphorylated derivatives, designed to increase solubility in hydrophilic media.

12. The composition of claim 11, wherein said androgen is present in an amount of from about 0.001 to about 0.1% by weight.

13. A method for treating or preventing Dry Eye Syndrome, elevated intraocular pressure, age-related maculopathy and/or age-related macular degeneration in a patient comprising topically applying to the eye of said patient an effective amount of a composition comprising, on a weight percent basis:

| | |
|---|---|
| Dibasic sodium phosphate | 0.05-1.0% |
| Sodium Chloride | 0.2-0.9% |
| Edetate disodium | 0.05-1.0% |
| Povidone | 0.05-2.0% |
| Poloxamer | 0.001-0.05% |
| Polyethylene glycol | 0.05-1.0% |
| Hydroxyethyl Cellulose | 0.05-1.0% |
| Purified water | q.s to 100% |
| HCl or NaOH | to adjust pH to pH 6-8. |

14. The method of claim 12, wherein the composition comprises, on a weight percent basis, about:

| | |
|---|---|
| Dibasic sodium phosphate | 0.3%; |
| Sodium Chloride | 0.6%; |
| Edetate disodium | 0.1%; |
| Povidone K-17 | 0.37%; |
| Poloxamer | 0.004%, |
| Polyethlyene glycol | 0.12%; |
| Hydroxyethyl Cellulose | 0.2%, |
| Purified water | q.s to 100%; |
| HCl or NaOH | to adjust pH to pH 6-8. |

15. The method of claim 12, wherein the composition further comprising one or more preservatives selected from the group consisting of methylparaben, propylparaben, benzalkonium chloride and phenoxyethanol.

16. The method of claim 13, wherein said composition further comprises one or more active ingredients selected from the group consisting of 17-β-estradiol, an androgen, or derivative thereof.

17. An artificial tear composition consisting essentially of, on a weight percent basis:

| | |
|---|---|
| Dibasic sodium phosphate | 0.05-1.0% |
| Sodium Chloride | 0.2-0.9% |
| Edetate disodium | 0.05-1.0% |
| Povidone | 0.05-2.0% |
| Poloxamer | 0.001-0.05% |
| Polyethylene glycol | 0.05-1.0% |
| Hydroxyethyl Cellulose | 0.05-1.0% |
| Purified water | q.s to 100% |
| HCl or NaOH | to adjust pH to pH 6-8. |

18. The artificial tear composition of claim 17, consisting essentialy of, on a weight percent basis, about:

| | |
|---|---|
| Dibasic sodium phosphate | 0.3%; |
| Sodium Chloride | 0.6%; |
| Edetate disodium | 0.1%; |
| Povidone K-17 | 0.37%; |
| Poloxamer | 0.004%, |
| Polyethlyene glycol | 0.12%; |
| Hydroxyethyl Cellulose F | 0.2%, |
| Purified water | q.s to 100%; |
| HCl or NaOH | to adjust pH to pH 6-8. |

19. The artificial tear composition of claim 18, further comprising one or more preservatives selected from the group consisting of methylparaben, propylparaben, benzalkonium chloride and phenoxyethanol.
